(19) United States
(12) Patent Application Publication
(10) Pub. No.: US 2004/0191327 A1
(43) Pub. Date: Sep. 30, 2004

(54) Method of treating and/or preventing asthma using natural compound luteolin

(75) Inventors: Balaram Ghosh, Delhi (IN); Moumita Das, Delhi (IN); Arjun Ram, Delhi (IN)

Correspondence Address: WOMBLE CARLYLE SANDRIDGE & RICE, PLLC, P.O. BOX 7037, ATLANTA, GA, 30357-0037, US

(73) Assignee: Council of Scientific and Industrial Research, New Delhi (IN)

(21) Appl. No.: 10/698,863

(22) Filed: Nov. 3, 2003

(30) Foreign Application Priority Data
Mar. 24, 2003 (WO) PCT/IN03/00072

(51) Int. Cl. A61K 35/78; A61K 35/12

(52) U.S. Cl. 424/522

(57) ABSTRACT

The present invention relates to a method of preventing and/or treating asthma in animals including humans using natural compound Luteolin, said method comprising administering therapeutically effective dose of the Luteolin and leading to level of IFN-gamma increasing to normal level, levels of IL-5, IL-4, and IgE decreasing to normal level, and further, said method inhibits airway constriction, and airway hyperreactivity.

### FIELD OF THE PRESENT INVENTION

The present invention relates to a method of preventing and/or treating asthma in animals including humans using natural compound Luteolin, said method comprising administering therapeutically effective dose of the Luteolin and leading to level of IFN-gamma increasing to normal level, levels of IL-5, IL-4, and IgE decreasing to normal level, and further, said method inhibits airway constriction, and airway hyperreactivity.

### BACKGROUND OF THE PRESENT INVENTION

Luteolin is a naturally occurring flavonoid, which has been attributed with anti-inflammatory (Mann et al., 1984; Xagorari et al., 2001) and anti-allergic properties (Mann et al., 1984). In vitro studies have elucidated some of the molecular mechanisms by which Luteolin modulates the inflammatory response. Luteolin has been reported to inhibit: a) the release of mediators like leukotrienes and prostaglandin's (Xagorari et al., 2001), b) the production of the proinflammatory cytokine, IL-5 (Park et al., 1999) and c) the process of signal transudation leading to the inhibition of nuclear transcription factor-kappa B (NF-KB mediated gene expression (Xagorari et al., 2001). In addition, Luteolin suppresses the expression of TNF-gamma. induced ICAM molecules on endothelial cells (Shimoi et al., 2000). Based on these evidences, we postulated that Luteolin might have a preventive and/or therapeutic role in attenuating asthma.

Asthma is an inflammatory disease of the airways and is characterized by difficulty in breathing due to constriction of smooth muscles of the bronchi as a result of inflammation. The development of the disease is mediated by cytokines- IL-4, IL-5, IgE, eosinophils and various mediators, e.g. histamine, leukotrienes and others (Abbas et al., 1994; Weiss et al., 1993) all of which lead to the symptoms of asthma. On the other hand, IFN-gamma inhibits this process (Barnes, 2000). The current focus in managing asthma is the control of inflammation using anti-asthmatic drugs with lower or negligible side effects (Barnes, 1999). Search is going on for novel drugs, especially of natural origin, as they may have negligible side effects.

Keeping in view of the above properties, we examined Luteolin for its anti-asthmatic activity using a mouse model of asthma. Till date there is no direct in-vivo experiment which demonstrated the effect of Luteolin on asthmatic features either in human or animal model. Novelty of the invention is in first in-vivo demonstration of Luteolin for alleviation the characteristic features of asthma produced in mouse such as allergen-induced early airway response (EAR) and late airway response (LAR).

### OBJECTS OF THE PRESENT INVENTION

The main object of the present invention is to develop a method of preventing and/or treating asthma in animals including humans using natural compound Luteolin.

Another main object of the present invention is to develop a method of treating asthma wherein, the compound Luteolin shows no side effects.

Another main object of the present invention is to develop a method of treating asthma wherein, the compound Luteolin is administered orally.

Yet another main object of the present invention is to develop a method of treating asthma using compound Luteolin wherein, the development of asthmatic features comprising Early Airway Response (EAR) and Late Airway Response (LAR) are prevented.

Still another main object of the present invention is to develop a method of treating asthma using compound Luteolin wherein, level of IFN-gamma increases to normal level.

Still another main object of the present invention is to develop a method of treating asthma using compound Luteolin wherein, level of IL-5 decreases to normal level.

Still another main object of the present invention is to develop a method of treating asthma using compound Luteolin wherein, level of IL-4 decreases to normal level.

Still another main object of the present invention is to develop a method of treating asthma using compound Luteolin wherein, level of IgE decreases to normal level.

Still another main object of the present invention is to develop a method of treating asthma using compound Luteolin wherein, compound Luteolin inhibits airway constriction.

Still another main object of the present invention is to develop a method of treating asthma using compound Luteolin wherein, compound Luteolin inhibits airway hyperactivity.

### SUMMARY OF THE PRESENT INVENTION

The present invention relates to a method of preventing and/or treating asthma in animals including humans using natural compound Luteolin, said method comprising administering therapeutically effective dose of the Luteolin and leading to level of IFN-gamma increasing to normal level, levels of IL-5, IL-4, and IgE decreasing to normal level, and further, said method inhibits airway constriction, and airway hyperreactivity.

### DETAILED DESCRIPTION OF THE PRESENT INVENTION

Accordingly, the present invention relates to a method of preventing and/or treating asthma in animals including humans using natural compound Luteolin, said method comprising administering therapeutically effective dose of the Luteolin and leading to level of IFN-gamma increasing to normal level, levels of IL-5, IL-4, and IgE decreasing to normal level, and further, said method inhibits airway constriction, and airway hyperreactivity.

In an embodiment of the present invention, a method of preventing and/or treating asthma in animals including humans using natural compound Luteolin, said method comprising administering therapeutically effective dose of the Luteolin.

In another embodiment of the present invention, wherein the compound Luteolin shows no side effects.

In yet another embodiment of the present invention, wherein the compound Luteolin is administered orally.

In still another embodiment of the present invention, wherein the development of asthmatic features comprising Early Airway Response (EAR) and Late Airway Response (LAR) are prevented.

In still another embodiment of the present invention, wherein level of IFN-gamma increases to normal level.

In still another embodiment of the present invention, wherein level of IL-5 decreases to normal level.

In still another embodiment of the present invention, wherein level of IL-4 decreases to normal level.

In still another embodiment of the present invention, wherein level of IgE decreases to normal level.

In still another embodiment of the present invention, wherein the concentration of compound Luteolin is ranging between 0.1 to 10 mg/kg body weights.

In still another embodiment of the present invention, wherein the concentration of compound Luteolin is ranging between 1 mg/kg body weights.

In still another embodiment of the present invention, wherein the duration of administering compound Luteolin is ranging between 5 to 10 days.

In still another embodiment of the present invention, wherein compound Luteolin inhibits airway constriction.

In still another embodiment of the present invention, wherein compound Luteolin inhibits airway hyperactivity.

### BRIEF DESCRIPTION OF ACCOMPANYING DRAWING

FIG. 1 shows structure of Luteolin

FIG. 2 shows the protocol for the sensitization and Luteolin treatment to mice.

Protocol A: The mice were given three i.p. Injections of 10 μg ovalbumin adsorbed on 2 mg alum on 0, 7 and 14 days and then subjected to OVA aerosol inhalation for five consecutive days from 19^{th} to 23^{rd} day of the experiment. On 24^{th} day SGaw measurements were performed after OVA challenge in the plethysmograph and airway reactivity to methacholine was measured after 24 hours. In case of curative study (Protocol B), sensitized mice were fed with 1 mg/kg body weight of Luteolin for one week (days 26-32) and the effects on SGaw and airway reactivity were evaluated.

FIG. 3 shows the effect of Luteolin during and after sensitization period on specific airway conductance, SGaw. Mice were fed with Luteolin for a period of 23 days during sensitization and for one week after sensitization. SGaw levels were measured before and after OVA aerosol challenge. * indicates significant difference from the sensitized group.

FIG. 4 shows the effect of feeding Luteolin during sensitization period on the development of airway hyperreactivity. Mice were fed with Luteolin during and after sensitization period and airway reactivity (MCh PD₃₅) was measured as described in Example 5. * indicates significant difference from the sensitized group.

FIG. 5 shows the effect of oral Luteolin treatment on OVA-specific IgE levels in serum of mice. * indicates significant difference from the sensitized group.

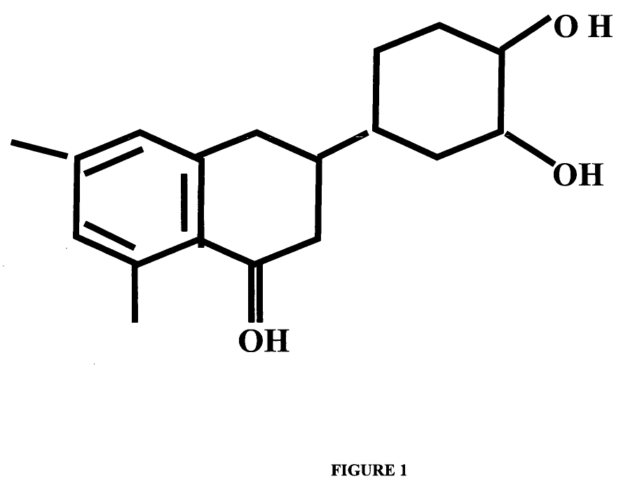

In still another embodiment of the present invention, wherein the existing anti-asthmatic drugs, particularly steroids, have many side effects. There is intense need to develop certain non-steroidal anti-asthmatic drugs preferably of natural origin. In this context, Luteolin (FIG. 1), a plant based natural non-steroidal anti-inflammatory compound, was tested for the anti-asthmatic activity using mouse model of asthma.

In still another embodiment of the present invention, wherein administration of pharmacologically effective dose of Luteolin to mice during sensitization prevented the development of both the asthmatic features (EAR and LAR). This finding showed a preventive effect of Luteolin on the development of asthma.

The present invention also showed that Luteolin, when administered orally to the animals already showing impaired airways features, alleviated the existing impaired features. Luteolin has been found to increase IFN-□ levels and attenuated IL-5 levels in the bronchoalveolar lavage (BAL) fluid. The allergen-specific IgE levels in the sera samples were also reduced significantly.

In still another embodiment of the present invention, wherein accordingly, present invention relates to novel use of Luteolin as an anti-asthmatic agent and the method of use comprises of:

a. sensitizing the animals by an antigenic protein to induce characteristic asthmatic features,

b. estimating asthmatic features prior to, during and after sensitization of the animals,

c. Administering pharmacologically active concentration of a solution of Luteolin to healthy animals during and after sensitization,

d. measuring immunological features in the sacrificed animals after step (b) and (c).

In still another embodiment of the present invention, wherein the animal model used may be selected from BALB/c mice, rabbits and guinea pigs.

In another embodiment to the invention the protein for sensitizing the animals may be administered through intraperitoneally injection or aerosol inhalation routes.

In yet another embodiment to the invention, the protein solution in normal saline used for sensitization may be selected from ovalbumin, bovine serum albumin or any other antigenic protein, in a concentration ranging from 10-100 μg per injection or 1-5% for inhalation by aerosol in normal saline.

In still another embodiment to the invention, luteolin may be administered orally to the animals in the concentration range of 0.1 to 10 mg/kg body weight.

In yet another embodiment, the asthmatic features may be estimated by known methods of measuring specific airway conductance or specific airway resistance.

In still another embodiment to the invention the immunological features may be measured by estimating IgE, IFN-γ, IL-4 and IL-5 levels by known methods.

In still another embodiment of the present invention, wherein asthma is an inflammatory disease of the airways, which affects millions of people worldwide. The disease is reaching epidemic proportions and young lives are increasingly rendered unproductive. Asthma is characterized by difficulty in breathing due to constriction of smooth muscles of the bronchi as a result of inflammation. It is characterized by elevated levels of immunoglobulin E in the blood and infiltration of eosinophils into the airways. The development of the disease is mediated by cytokines- IL-4 and IL-5, IgE, eosinophils and various other mediators e.g. leukotrienes, cyclooxygenase products, phospholipases all of which lead to the symptoms of asthma (Abbas et. al., 1994, Weiss et. al., 1993). In contrast, IFN-□ inhibits this process (Barnes, 2000).

The current focus in managing asthma is to control inflammation using anti-asthmatic drugs with low side effects (Barnes, 1999). There is a need for novel drugs for the treatment of asthma, which may have fewer side effects.

In this context, Luteolin was tested on mouse model of asthma. Mice were sensitized with intraperitoneal and aerosol inhalation of OVA to develop the characteristic features of asthma such as allergen induced early airway response (EAR) and late airway response (LAR). These asthmatic features were characterized by measuring airway calibre in the term of specific airway conductance (SGaw) by a non-invasive technique, dual-chamber whole body plethysmography. After developing the characteristic features (EAR and LAR) in mouse, the compound, Luteolin was given orally during whole sensitization period to test the preventive effect on the development of asthmatic features. To examine the therapeutic effect of Luteolin on the asthmatic features, it was fed for one week to mice after sensitization and confirming their asthmatic features.

After testing the compound for the preventive as well as for therapeutic effects in intact conscious mice, mice were sacrificed for collecting the blood and bronchoalveolar lavage (BAL) fluid to measure the levels of IgE, cytokines IL-4, IL-5 and IFN-γ. Ovalbumin specific IgE levels in the sera and the levels of IL-4, IL-5 and IFN-γ in the BAL fluid were measured by enzyme linked ImmunoSorbent assay (ELISA) kits.

In still another embodiment of the present invention, wherein the present invention provides an effective compound for preventing the development of the characteristic features of asthma in an animal. For example, there was prevention of the development of airway constriction and airway reactivity in mice treated orally with Luteolin during sensitization.

In still another embodiment of the present invention, wherein the present invention also demonstrates that Luteolin is effective when given to mice after sensitization i.e., after developing airway hyperactivity. Luteolin administered orally for one week to airway hyperactive animals, inhibited both allergen induced airway constriction and airway hyper reactivity to methacholine. This showed the therapeutic potential of this compound.

The present invention also showed that Luteolin reduces IgE in the serum and IL-5 in the BAL fluid, which is favorable for improving the impaired airways features (EAR and LAR).

Luteolin administration to the mice both during sensitization as well as after sensitization, increased significantly the levels of IFN-gamma in the BAL fluid. This finding suggests that Luteolin inhibits inflammation by administering its pharmacologically effective dose.

The effective dose was found to be 1 mg/kg body weight.

TABLE I
Luteolin up regulates IFN-gamma levels and
decreases IL-5 levels in the BALF.

| Treatment | IFN-γ(pg/ml) | IL-4 (pg/ml) | IFN-γ: IL-4 | IL-5 (pg/ml) |
|---|---|---|---|---|
| Sham- | 2650 ± 50 | 130 ± 10 | 9.2 ± 5.1 | 118 ± 19 |
| sensitized | | | | |
| Sensitized | 81 ± 3.8 | 171 ± 23.8 | 0.5 ± 1.0 | 149 ± 3.5 |
| Luteolin | 3533 ± 427** | 136 ± 4.0 | 26 ± 2.7** | 73 ± 13.4* |
| (0.1 mg/kg)^{a} | | | | |
| Luteolin | 2652 ± 114.3** | 169 ± 13.9 | 16 ± 1.3** | 43 ± 3.5* |
| (1.0 mg/kg)^{a} | | | | |
| Luteolin | 1247 ± 412.5** | 128 ± 8.8 | 13 ± 2.5** | 235 ± 66.4 |
| (10.0 mg/kg)^{a} | | | | |
| Luteolin | 2067 ± 233.3** | 130 ± 5.0 | 16 ± 2.2** | 44 ± 2.3* |
| (1.0 mg/kg)^{b} | | | | |

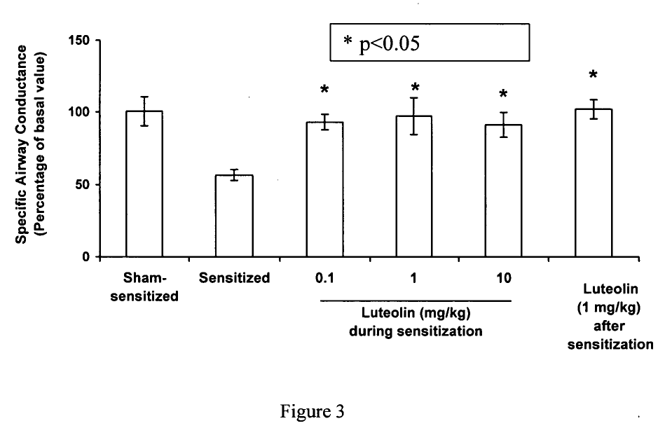

Experiments were performed with six groups of mice (n=6) as described in FIG. 3 legend and the levels of IFN-γ, IL-4 and IL-5 were measured in the BALF. The values are a mean±SEM of six mice in each group. Mann Whitney U test was used to determine the significant differences between the sensitized- and Luteolin treated groups. * p <0.05, ** p<0.01. ‘a’ denotes treatment during sensitization; ‘b’ denotes treatment after sensitization.

The following examples are given for the purpose of illustrating various embodiments of the inventions and are not meant to limit the present invention in any fashion.

### EXAMPLE 1

Animals' Sensitization:

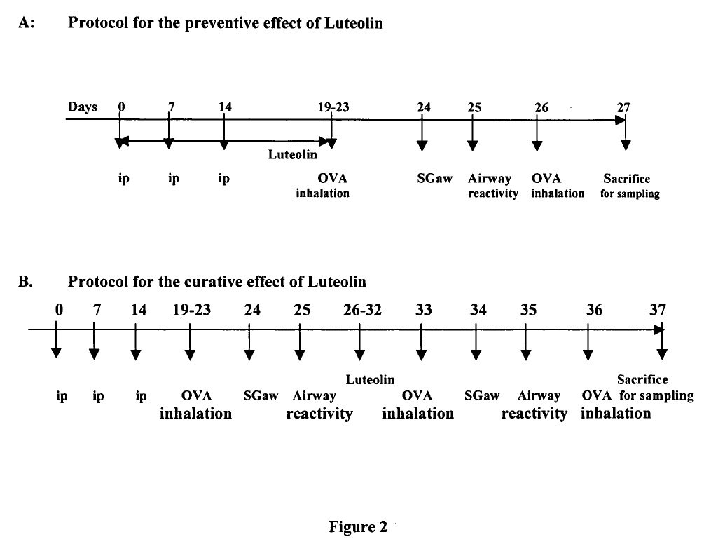

Mice were immunized/sensitized with or without (sham-sensitized) 0.2 ml of 10 μg ovalbumin (Sigma Chemicals Co. St Louis, Mo., USA) and 2 mg aluminum hydroxide intraperitoneally on days 0,7 and 14 using a modified protocol previously described by Sakai et al (FIG. 2). Five days after the final i.p injection, the mice were subjected to aerosolized OVA (5%) or phosphate buffered saline (PBS) (for sham-sensitized group) inhalation for 20 minutes daily beginning from day 19 to day 23 (FIG. 2). Mice were placed in a plexiglas chamber (20×20×20 cm dimensions) and exposed to an aerosol generated from a nebulizer (de Vilbiss, USA) with airflow of 41/min.

### EXAMPLE 2

Treatment of Mice with Luteolin:

To study the preventive effect, Luteolin (dissolved in 50% hydro-alcohol) (Sigma Chemicals Co. St Louis, Mo., USA) (0.1, 1.0, and 10.0 mg/kg body weight, 20 □| volume) or vehicle (i.e., 50% hydro-alcohol) was administered orally to each group of mice daily, starting from the first day of sensitization (FIG. 2A). To study the curative effect, mice were first sensitized as described before (Example 1) followed by oral Luteolin (1.0 mg/kg body weight) treatment daily from day 26 to 32 (FIG. 2B).

### EXAMPLE 3

Measurement of Airways Calibre:

Airway calibre was measured in the term of specific airway conductance (SGaw) using a dual-chamber whole body plethysmograph similar to the method for guinea pigs (Agrawal, 1981) with some modifications. The dual-chamber whole body plethysmograph was designed in our laboratory to suit the size of mouse. The value of SGaw was calculated as described by Agrawal (1981).

### EXAMPLE 4

Luteolin Inhibits Acute OVA-Induced Airway Constriction:

Airway constriction of mice was determined in terms of fall of SGaw due to OVA aerosol challenge. SGaw was measured as described in Example 3. To determine the preventive effect of Luteolin, mice were dosed with Luteolin (0.1, 1.0 and 10.0 mg/kg body weight) or vehicle during sensitization, as described in the Example 2. Following challenge, OVA-sensitized mice treated with vehicle showed a 43% fall in specific airway conductance (SGaw) as compared to their basal values, whereas sham-sensitized mice showed no change (FIG. 3). Interestingly, treatment with Luteolin markedly prevented OVA-induced decrease in SGaw. The dose of 0.1 mg/kg Luteolin markedly reduced the fall in SGaw induced by OVA and only a 7% decrease was recorded (p<0.01). Further increase of the doses did not show any greater reduction in OVA-induced decreases in SGaw (p<0.01). To examine the curative effect of Luteolin, mice were first sensitized and then treated with Luteolin (1.0 mg/kg) for one week (Example 2). Luteolin was found to reverse OVA-induced decrease in SGaw (FIG. 3).

### EXAMPLE 5:

Luteolin Reduces Airway Reactivity to Methacholine (MCh):

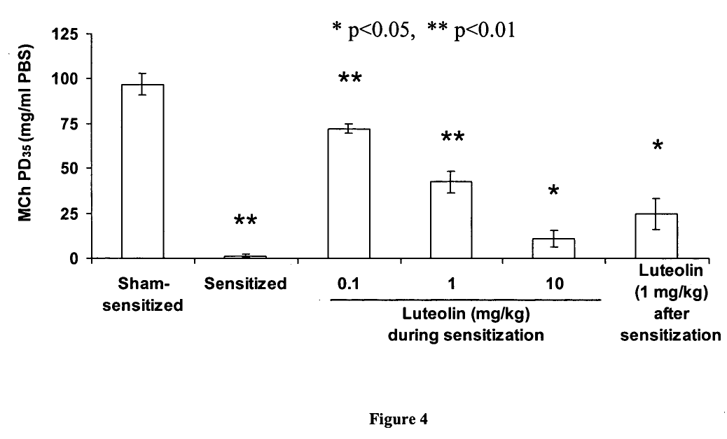

Airway reactivity to methacholine was determined by measuring the concentration of inhaled methacholine that produced 35% fall in SGaw (MCh PD₃₅) in intact mouse sitting in the body plethysmograph. Aerosol of different concentrations of methacholine (3.1, 6.25, 12.5, 50, 100 mg/ml) were given for 60 seconds. MCh PD₃₅ values were determined in sham-sensitized, sensitized and Luteolin treated mice during and after sensitization. As shown in FIG. 4, there was a significant fall in MCh PD₃₅ values (1.3±0.5 mg/ml PBS) following OVA challenge in sensitized mice compared to normal mice (97±1.0 mg/ml, p<0.01). Treatment with Luteolin (0.1, 1.0 and 10.0 mg/kg during sensitization) markedly attenuated the development of airway hyperreactivity to methacholine. MCh PD₃₅ values increased following Luteolin treatment to 72.1±1.9, 42.4±4.2 and 10.9±4.8 mg/ml, respectively (p<0.01). In addition, when the mice were first sensitized and then treated with Luteolin (1.0 mg/kg) for one week, MCh PD₃₅ values increased significantly (p<0.01) suggesting that Luteolin inhibited the development of OVA-induced airway hyperreactivity.

### EXAMPLE 6:

Luteolin Treatment during and after Sensitization Reduces Serum IgE Levels.

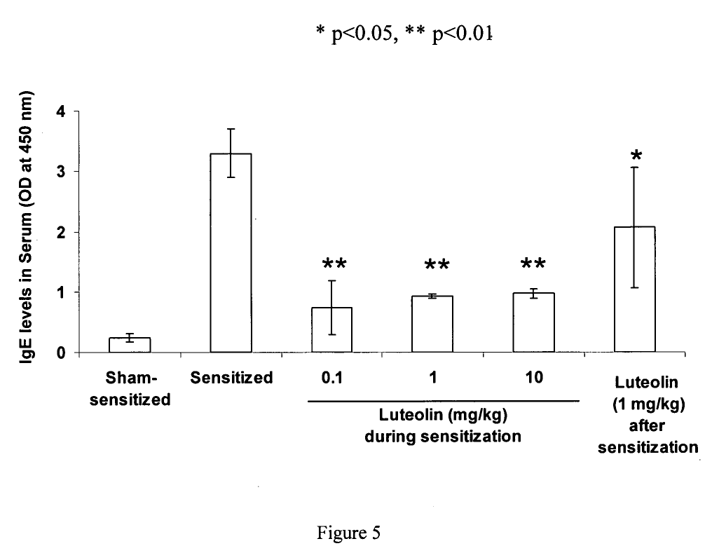

OVA-specific IgE levels in the serum were measured by enzyme linked ImmunoSorbent assay (ELISA), To investigate the effect of Luteolin on serum IgE levels, we compared the levels of the OVA-specific IgE in serum of mice treated with vehicle or with Luteolin (0.1, 1.0 and 10.0 mg/kg) during sensitization and in sham-sensitized mice. As shown in FIG. 5, the IgE values increased in sensitized mice when compared to sham-sensitized mice (3.3±0.4 versus 0.24±0.07; p<0.05). Luteolin was found to decrease the serum IgE levels in sensitized mice when compared with vehicle treated sensitized group (0.74±0.4, 0.93±0.03, 0.97±0.07 following doses of 0.1, 1.0 and 10.0 mg/kg respectively versus 3.3±0.4 in sensitized, vehicle treated mice; p<0.05). When serum IgE levels in mice, first sensitized and then treated with Luteolin (1.0 mg/kg) for a week, were measured, the IgE level was found to be decreased (2.07±1) as compared to sensitized mice (p<0.05).

### EXAMPLE 7:

Luteolin Increases the Ratio of IFN-γ to IL-4 and Decreases IL-5 in the BAL Fluid.

To investigate the levels of IL-4 and IFN-□ in the BALF, we measured the cytokine levels by ELISA as per manufacturer's protocol, and compared between the different groups. As shown in Table 1, serum levels of IFN-γ were significantly elevated in OVA-sensitized mice treated with Luteolin, whereas in the untreated OVA-sensitized mice, IgE levels were reduced. In the Luteolin-treated group, there was an increase in the ratio of IFN-γ/IL-4 (25.8±2.7, 15.5±1.3, 13.2±2.5 following doses of 0.1, 1.0 and 10.0 mg/kg Luteolin, respectively as compared to 0.48±0.1 in sensitized, vehicle treated mice) (p<0.01) (Table 1). In the mice first sensitized and then treated with Luteolin (1.0 mg/kg) for one week, the ratio of IFN-γ/IL-4 was also increased (16±2.2) (p<0.01) as compared to sensitized, vehicle treated mice. This increase in the IFN-γ/IL-4 ratio was due to the increase in the IFN-γ levels. We also measured the concentration of IL-5 in the BALF by ELISA in the different groups (Table 1). In the Luteolin-treated group, a decrease in the levels of IL-5 was seen (73.3±13.4 and 43.3±3.5 following doses of 0.1 and 1.0 mg/kg Luteolin respectively as compared to 148.5±3.5 in sensitized, vehicle treated mice)(Table 1). In the sensitized mice treated with Luteolin (1.0 mg/kg) for one week, the IL-5 level was decreased (43.7±2.3) as compared to sensitized, vehicle treated mice.

Advantages of the Present Invention

1. This is the first demonstration that Luteolin inhibits the characteristic features of asthma produced in an animal model and can be used for development of effective drugs for asthma therapy.

2. Luteolin, being a plant based natural non-steroidal compound, may have lesser side effects than the existing therapeutic steroids.

3. This compound is readily available.

4. The use of Luteolin may not be restricted only to anti-asthmatic agent, but to other inflammatory conditions where elevations of IgE and IL-5, and reduction in IFN-gamma levels play significant roles.

1. A method of preventing and/or treating asthma in animals including humans using natural compound Luteolin, said method comprising administering therapeutically effective dose of the Luteolin.

2. A method as claimed in claim 1, wherein the compound Luteolin shows no side effects.

3. A method as claimed in claim 1, wherein the compound Luteolin is administered orally.

4. A method as claimed in claim 1, wherein the development of asthmatic features comprising Early Airway Response (EAR) and Late Airway Response (LAR) are prevented.

5. A method as claimed in claim 1, wherein level of IFN-gamma increases to normal level.

6. A method as claimed in claim 1, wherein level of IL-5 decreases to normal level.

7. A method as claimed in claim 1, wherein level of IL-4 decreases to normal level.

8. A method as claimed in claim 1, wherein level of IgE decreases to normal level.

9. A method as claimed in claim 1, wherein the concentration of compound Luteolin is ranging between 0.1 to 10 mg/kg body weights.

10. A method as claimed in claim 1, wherein the concentration of compound Luteolin is ranging between 1 mg/kg body weights.

11. A method as claimed in claim 1, wherein the duration of administering compound Luteolin is ranging between 5 to 10 days.

12. A method as claimed in claim 1, wherein compound Luteolin inhibits airway constriction.

13. A method as claimed in claim 1, wherein compound Luteolin inhibits airway hyperactivity.
